(19) United States
(12) Patent Application Publication
(10) Pub. No.: US 2010/0042043 A1
(43) Pub. Date: Feb. 18, 2010

(54) AUTOMATIC DRUG ADMINISTRATION WITH REDUCED POWER CONSUMPTION

(75) Inventors: Hendrika Cecilia Krijnsen, Eindhoven (NL); Ventzeslav Petrov Iordanov, Eindhoven (NL); Anna M. Janner, Eindhoven (NL); Michel Paul Barbara Van Bruggen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/593,026

(22) PCT Filed: Mar. 20, 2008
(86) PCT No.: PCT/IB08/51065

§ 371 Date: Sep. 25, 2009

(30) Foreign Application Priority Data
Mar. 27, 2007 (EP) 07105000.9

(51) Int. Cl. A61M 5/168; A61K 9/22; G06F 1/32

(52) U.S. Cl. 604/66; 604/65; 604/891.1; 713/324

(57) ABSTRACT

It is described a drug delivery system (100) that is adapted to infuse at least one drug in a profile related to the status of a patient. The system comprises sensors (110), dedicated electronics (140) and software to monitor the patient's activity and maintain control over the drug-release profile. A control unit (120) of the system (100) is adapted to temporarily set at least one component (110, 120, 150) of the system (100) in an idle mode (265). This will result in particular in a reduction of power required by the system (100). The described system (100) enables a personalized treatment of patients by monitoring their individual activity status and coupling this information to a drug delivery following appropriate delivery profiles. The system (100) may be provided with a memory in order to store a time dependence of activity data. This time dependence may be used by physicians to monitor, support and adapt their therapy for the patients. The system (100) may be realized as a closed-loop system for monitoring and treatment of various diseases such as hemophilia A and Parkinson.

### FIELD OF INVENTION

The present invention relates to the field of delivering drugs to a patient. In particular the present invention relates to a system and to a method for automatically delivering drugs to the body of a patient in a correct dosage by appropriately controlling the operation of a drug delivery device.

### ART BACKGROUND

Traditionally, drug products are given via tablets or oral capsules. These are taken for example once a day in the morning or in the evening. This results in a bolus in the blood drug level after intake and a subsequent exponential decline of the drug in the blood over time. More advanced systems take into account timing of medication to increase drug efficacy by advising the patient to preferably administer the drug at some time of day, for example in the evening after having diner. At some time during the day, prior to the next administration, the blood drug level may be below a lower therapeutic limit and the patient may experience symptoms and/or disease progression.

More and more prophylactic treatment or maintenance therapy is applied in order to combat a disease already at the beginning or before the disease effectively appears. This therapy comprises the administration of lower amounts of drugs at a higher frequency. This results in a smoother, more constant blood drug level being within the therapeutic window.

WO 2006/096654 A2 discloses a microjet fluid delivery system including a reservoir, a delivery actuator and a delivery nozzle having an exit orifice with a diameter between about 1 μm and about 500 μm. The delivery actuator is configured to deliver a quantity of fluid contained in the reservoir into the tissue of a patient. Thereby the fluid has a pre-determined velocity leading to a deposition of the fluid in a desired depth. The quantity of fluid may contain one or more therapeutic agents such as medications, drugs, bio-reactive agents, etc. The delivery actuator may also be configured to repeatedly deliver a quantity of the fluid contained in the reservoir through the nozzle at pre-determined intervals. The delivery actuator may be coupled to a sensor such that the quantity of delivered fluid can depend on a signal provided by the sensor. The sensor may be a biosensor selected from one or more of a pressure sensor, a density sensor, a chemical sensor and/or an electrical sensor. The biosensor may be located internally or externally of a patient's body.

WO2004/012796 A1 discloses a delivery device suitable for treatment of diseases or conditions in which a drug has to be applied through the skin of a patient. The delivery device is provided for delivering a liquid drug into the body of a patient. The delivery device comprises a reservoir having, in a situation of use, an outlet, an amount of a liquid drug contained in the reservoir, expelling means for expelling the drug out of the reservoir through the outlet, and actuating means for actuating the expelling means. Upon actuation the expelling means is adapted for expelling the drug contained in the reservoir during a period of approximately 7-9 hours. The disclosed drug delivery device has the disadvantage that, once activated, there is no further control of the amount of drug, which is going to be delivered to the patient.

US 2003/104982 A1 discloses a system and a method for dosing a hormone regulating the blood glucose, especially insulin of a diabetic patient. In order to improve the administration of the hormone, the invention provides the following characteristic combination: (a) a measuring device to detect measured values correlatable with blood sugar, (b) a controlling means comprising a controller and a hormone dosing device for supplying a hormone dosage, (c) a pilot control device acting on the hormone fine dosage controlling means for performing a coarse pre-control in accordance with at least one influence variable that influences blood glucose.

There may be a need for providing a system for delivering drugs into the body of a patient, which system (a) allows for an appropriate control of the amount of drug being delivered to a patient and (b) can be operated in an effective manner.

### SUMMARY OF THE INVENTION

This need may be met by the subject matter according to the independent claims. Advantageous embodiments of the present invention are described by the dependent claims. According to a first aspect of the invention there is provided a system for delivering at least one drug to the body of a patient. The provided system comprises (a) a measurement device for measuring at least one measurement value, which is indicative for the status of the patient, (b) a control unit, which is coupled to the measurement device and which is adapted to determine a control signal based on the measurement value, and (c) a drug delivery device, which is coupled to the control unit and which is adapted to deliver the drug to the patient's body based on the control signal. The control unit is adapted to temporarily set at least one component of the system in an idle mode.

This aspect of the invention is based on the idea that the power consumption of the described system can effectively be reduced by temporarily setting the measurement device, the control unit and/or the drug delivery device in an idle mode. In the provided drug delivery system this is managed by the control unit.

In case also the control unit sets itself into an idle mode it is clear that the control unit is not switched off completely. The control unit can be activated again by a predetermined lapse of time, which is measured by a permanently running internal clock of the control unit. Alternatively or in addition the control unit can be activated by receiving an external activation signal. This activation signal may be generated by the patient and/or by an external patient monitoring system.

The described automatic drug delivery system may provide the advantage, that the drug administration can be carried out depending on the status of the patient's body. In particular the activity status representing the current physical activity of the patient and/or a predetermined daily rhythm of the patient may be taken into account for the amount and/or the temporal run of the drug administration.

The described drug delivery system may be used for administering also more than one type of drug. This may provide a great benefit for patients suffering from a disease, which (a) requires a treatment with a first drug when a certain chemical and/or physical value of the patient is in a hyper-range, i.e. compared to a normal range the value is too high and which (b) requires a treatment with a second drug when this chemical and/or physical value is in a hypo-range, i.e. compared to the normal range the value is too low. Such a situation occurs for instance for a patient suffering from diabetes type I. Thereby, when the patient is in a hyper-glycemia state insulin has to be administered. By contrast thereto, when the patient is in a hypo-glycemia state glucose has to be administered.

In this respect it is mentioned that In particular if more than one type of drug is administered the drug delivery system may comprise a corresponding number of drug delivery devices. This may provide the advantage that the applied dosage of each type of drug can be controlled very precisely. However, it may also be possible that one drug delivery devices may be used for providing two or more different type of drugs.

In many cases the required amount of a drug administration to a patient's body strongly depends on the activity status of the patient. By measuring the activity status of the patient a knowledge of this dependency can be used in order to optimally adjust the drug dose. This may provide the advantage that a disease the patient is suffering from can be treated in an optimal way. Therefore, depending on the particular disease the comfort of the patient and/or the expected lifetime of the patient may be increased. The optimal drug dosage further provides the advantage that the amount of drug being necessary for treating a disease can be reduced such that the effective medical costs for the treatment can also be reduced.

The system may be realized by means of an integral device, which is adapted to accomplish the function of at least two of the above-mentioned devices, namely the measurement device, the control unit and/or the drug delivery device. By contrast thereto, the system may also be realized by separate devices, which are coupled with each other in an appropriate way. The coupling can be realized by means of a wired or a wireless connection.

It has to be mentioned that the described system for delivering drugs is applicable not only for human patient's but also for animals. In particular in affluent societies there is an increasing demand for a treatment of animals respectively domestic animals, which suffer from similar diseases as human beings.

The measurement value may be any physical or chemical quantity, which can be employed as an indicative for the current status of the patient. The accuracy of the determination of the current status can be increased by combining different types of quantities to be measured. However, also different measurement values representing the same physical or chemical quantity can be combined in particular when these measurement values are obtained from different regions of the patient's body.

The time dependence of the operation of the drug delivery device may follow a pulsed operation of the measurement device. However, the measurement frequency may be lower than drug administration frequency for drugs having a short elimination half life in comparison to the stability of the measured parameter. This holds for instance for HbA1c as an example for diabetes management. In other words, if the measurement frequency is lower than the administration frequency, this means that the drug delivery may continue even when the measurement device is switched off or is at least not active.

Apart from reducing the power consumption this may provide the advantage that the amount of drugs being needed for treating a disease might also be reduced. Whereas prophylaxis already reduces medication costs compared to bolus administrations, this reduces the costs even further.

The operation of the complete system may be based on a pharmacokinetic model that is tuned to the individual patient. Such a pharmacokinetic model describes the time dependency of a drug level concentration within a patient. It is clear that the time dependency of a drug level concentration strongly depends on the time and the amount of drug administration and patient's body parameters such as the distribution volume of a patient normalized to a certain dose, the quality of the respective drug and/or the stability of the drug. The reciprocal distribution volume may depend on the type of drug, the location of delivery within the patient's body, the lean body mass of the patient, the weight of the patient, the patient's body area and/or the type of species the drug is applied to.

In case of the disease hemophilia A, the actual FVIII level is based on the blood drug level which itself depends on prior administrations and pauses and on the endogenous FVIII production and level of FVIII antibodies. Alternatively, the drug delivery system may be adjusted to provide a standard dosage given at daytime at a certain administration frequency and a higher dosage at waking up.

According to an embodiment of the invention the control unit is adapted to control a sampling rate of the measurement device. This may provide the advantage that the sampling rate can be adjusted during operation of the drug delivering system in order to reach an optimal ratio between power consumption of the whole system and useful measurement data being provided by the measurement device. By increasing the sampling rate more measurement data will be available at the expense of the power consumption. By lowering the sampling rate both the number of available measurement data and the power consumption will be reduced.

According to a further embodiment of the invention the measurement device comprises an acceleration sensor, in particular a three-dimensional acceleration sensor. The acceleration sensor may provide the advantage that it directly measures the motion of the patient. Thereby, in particular the activity status can be determined in a very reliable manner.

A three-dimensional (3D) acceleration sensor may be used for instance for determining the gravity direction. When the gravity direction is sensed as to be perpendicular to the body length it can be concluded that the patient is located in a lying posture. When this lying posture is maintained for a predetermined time period it can be further concluded that the patient is in a comparatively inactive status and the drug release dosage may be adjusted accordingly. Depending on the type of disease and the type of drug the drug dosage release can be increased or lowered when the activity status of the patient is low.

The described determination procedure of the positional state of the patient's body may for instance be used for patient's suffering from congenital coagulation defects such as hemophilia A. Presently the hemophilia A disease can be treated by applying a blood protein that is essential for clotting. Factor VIII is one prominent example of such a protein. Since the risk of injury of the body—and therefore the risk of bleeding—is significantly reduced when the patient is asleep, it may be economic, with regard to both saving drug and saving power of the drug delivery system, to turn off the drug delivery system for some time of day respectively the night when drugs are not required.

The described determination procedure of the positional state of the patient's body may also be advantageous for patients receiving human growth hormones. By contrast to the reduced delivery of a drug comprising Factor VIII proteins during the night, when the patient is asleep, the human growth hormone may only be delivered at nighttime in order to better mimic release in healthy persons and save energy.

An acceleration sensor may also be used for measuring tremor motions of a patient suffering from Parkinson's disease. By controlling the drug delivery device based on the strength and/or the frequency of the tremor motions a closed-loop system for treating the Parkinson's disease can be established. Therefore, the described system enables a personalized treatment of patients by directly coupling tremor sensing information to drug delivery following on-demand and/or programmed delivery profiles.

In particular when tremor motions are supposed to be sensed the measurement device should be placed at an appropriate position. Therefore, it can be advantageous that the measurement device on the one hand and the control unit respectively the drug delivery device on the other hand are separate preferably modular devices.

An acceleration sensor can also be used for sleep detection, which may be beneficial for the determination of the drug flux also in the case of Parkinson's disease. The required dose of e.g. dopamine receptor agonists can then be reduced significantly.

According to a further embodiment of the invention the measurement device comprises a magnetic field sensor.

By evaluating the time dependency of the strength and/or of the orientation of a magnetic field it is also possible to obtain a measure for the activity status of a patient. Thereby, the activity status may be obtained from evaluating the static posture of the patient and/or from evaluating the dynamic of changes of the strength and/or the orientation of the magnetic field such that information about the dynamic movement of the patient can be obtained. The magnetic field, which is used for determining the activity status may be represented by all kinds of magnetic fields which are existent in and around the patient. Since the sensitivity of magnetic sensors is quite high also the earth magnetic field may be sensed in order to obtain information about the static posture and/or the dynamic motion of the patient.

According to a further embodiment of the invention the measurement device comprises a temperature sensor. This means that also the body temperature might be used as a measure for the activity status of the patient's body. Thereby, the temperature can be measured in the core and/or at the surface of the patient's body. Also the temperature of the patient's skin can provide valuable information regarding the activity status.

According to a further embodiment of the invention the measurement device comprises a skin impedance sensor. This means that the electric resistivity may also be used as a measure for the activity status of the patient's body. Thereby, the resistance respectively the impedance can be measured under direct current and/or under alternating current conditions. The resistance may be affected in particular by the humidity and/or the salt concentration at the skin surface. Therefore, also the impedance of the patient's skin can provide valuable information regarding the patient's activity status.

According to a further embodiment of the invention the system further comprises a memory for storing a plurality of measurement values, which are indicative for the status of the patient. Thereby, the measurement values have been acquired at different times.

This may provide the advantage that a typical daily rhythm of the patient's activity in particular with respect to a diurnal or a nocturnal profile can be monitored. Thereby, time averaged activity data can be used as an objective measurement to get knowledge when the patient is asleep or at least at rest for some time.

Based on the daily rhythm, which can be measured for instance by measuring the core body temperature (alone or in combination with another measurement), the drug dosage may be adjusted. For example, when a hemophilic patient is at sleep corresponding to a low body core temperature, the delivery of FVIII may temporarily stop and continue when the patient awakes or just prior to the awakening of the patient.

By contrast thereto, when a patient, who receives human growth hormones, falls asleep, the drug delivery may commence and stop before the patient awakes.

In case of prophylactic treatment in hemophilia A, a FVIII drug dosage may be stopped for some time and may commence when activity goes up again. A simple calculation based on 8 h sleep per 24 h indicates a reduction in power consumption of 33% compared to continuous infusion or pulsatile infusion for example every half hour. This may provide the advantage that the patient does not need to have a fixed waking and sleeping time, but the system measures the body's rhythm and bases drug delivery by appropriately switching the drug delivery on and off. Preferably, in case of a hemorrhage, the measured activity on/off signal should be overruled because in that case an increased FVIII level is needed even during the sleep period. The use of activity direction measurement may be warranted or used in combination with various physical and/or chemical quantities to be measured. In particular the above-described temperature measurement of the body core temperature might be used for providing further information about the activity status of the patient. However, a halting FVIII drug release should be avoided at all cases when a patient has fallen or hurt himself so badly that he cannot move himself very much.

It has to be mentioned that a switching of the drug delivery device may also be prevented effectively if the patient can turn on a so-called “sleep mode” on the drug delivery device. This can ensure that the drug delivery device will not be switched off and drug delivery is guaranteed.

In case of patient suffering from Parkinson's disease the memory enables the recording of tremor motions as a function of time and a comparison between drug-delivery states with the patient evolving in a familiar environment such as the home of the patient. This may reduce an extra stress, which could influence the outcome of such types of tests.

Further, in case of a patient suffering from Parkinson's disease the memory may store data, which represent the daily rhythm of the patient's activity. Therefore, it is possible to initiate an enhanced drug delivery for instance already two hours before the patient wakes up. This may further improve the treatment of the patient' Parkinson disease.

According to a further embodiment of the invention the measurement device, the control unit and/or the drug delivery device is implantable into a patient's body. This may provide the advantage that (a) the activity status can be derived from a physical and/or a chemical quantity, which is present in the interior of the patient's body, and (b) the drug can be delivered to a location within the patient's body, which location might ensure for a strong drug response of the patient's body.

The system can be operated with different types of energy supplies such as an external magnetic and/or inductive actuation, an electrochemical actuation and an electro osmotic actuation. Further, in particular the drug delivery device may be operated with the energy stored in a compressed spring, which upon relaxing can drive a spring driven pump.

According to a further embodiment of the invention the control unit is adapted to allow for an interventional control ability. This may provide the advantage that the patient and/or a physician can temporarily manually adjust the drug level.

In addition to prophylaxis, it is sometimes necessary to temporarily provide higher drug levels than those used in steady state prophylaxis. This means that not only the constant release of drug is required to provide prophylaxis, but interventional controllability of the drug release amount may be needed in addition in order to temporarily increase the drug level. Thereby, the basal rate of drug release can be temporary adjusted according to the patient's respectively according to a caregiver's wish. In case of a hemophilic patient such a wish for a temporarily increase of the FVIII level may be existent for example if the patient intends to go sporting.

According to a further embodiment of the invention the system further comprises a radio transmitter, which is coupled to the control unit.

The provision of a radio transmitter may allow for a communication between the described drug delivery system and an external system providing control information such as for instance an interventional control signal. Further, measurement data may be received by the external system. The received measurement data may be evaluated in particular by a physician in order to obtain detailed information about the status of the patient's disease.

The radio transmitter may also be used for a wireless communication between the control unit and the measurement device and/or between the control unit and the drug delivery device. In this respect it should be clear that the provision of a radio transmitter is in particular advantageous if the described drug delivery system is implanted into the patient's body.

According to a further embodiment of the invention the system further comprises a battery monitoring circuit, which is adapted to monitor the state of a battery being used for powering the measurement device, the control unit and/or the drug delivery device. This may provide the advantage that the patient can be informed in due time before the battery runs dead. Therefore, there will be enough time to counteract a complete discharge of the battery either by charging the battery in a wireless manner for instance by inductive coupling or by replacing the battery with a fresh one.

According to a further embodiment of the invention the drug delivery device is adapted to deliver liquid type of medication. The drug delivery device may be driven by for example a step-motor based drug pump, a miniature piezo-driven drug pump and/or a pump based on switchable membranes. Depending on the specific application the pump may be an external ambulatory or an internal implantable device.

In case an implantable device is used an effective measure can be carried out in order to prevent unfavorable processes such as an encapsulation and/or the infiltration of body fluids into the device and in particular in a drug reservoir of the device. Such a measure is for instance the implementation of a very small constant drug release rate in between successive drug administration procedures. Preferably, the small constant drug release can be accomplished without using a battery of the system for instance by exploiting a pressure difference between the drug reservoir and the tissue of the patient's body. Such a pressure difference may be an osmotic pressure difference, which may be based for instance on a different salt concentration.

According to a further embodiment of the invention the drug delivery device is adapted to deliver solid type of medication. The medication may be for instance solid powder. In order to facilitate the administration the powder may be dispersed into a gas or liquid. When using a liquid for dispersing solid medication the above mentioned types of drug delivery device may be employed.

According to a further aspect of the invention there is provided a method for delivering drugs to the body of a patient by using a drug delivery system as described above. The method comprises (a) measuring at least one measurement value, which is indicative for the status of the patient, (b) determining a control signal based on the measurement value, (c) delivering the drug to the patient's body based on the control signal and (d) temporarily setting at least one component of the system in an idle mode.

This aspect of the invention is based on the idea that the power, which is needed for operating an automatic drug delivery system, can be effectively reduced by setting components of the drug delivery system into an idle mode. Of course this is done when these components are temporarily not needed. Further, if the drug delivery device is put into the idle mode the amount of drug, which will be delivered, can be effectively reduced by only administrating when needed. Thereby, the efficiency of the drug administration can be increased. It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG. 1 shows a simplified block diagram of a drug delivery system comprising a temperature sensor and a 3D posture sensor respectively a 3D acceleration sensor for monitoring the activity status of a patient.

FIG. 2 shows a diagram depicting the time dependency of the power consumption of a preferable operation mode of the drug delivery system depicted in FIG. 1, whereas the power consumption of the drug delivery device is not taken into account.

FIG. 3 shows a diagram depicting the time dependency of a patient's blood drug level during an intermediate halt of a drug release.

### DETAILED DESCRIPTION

The illustration in the drawing is schematic.

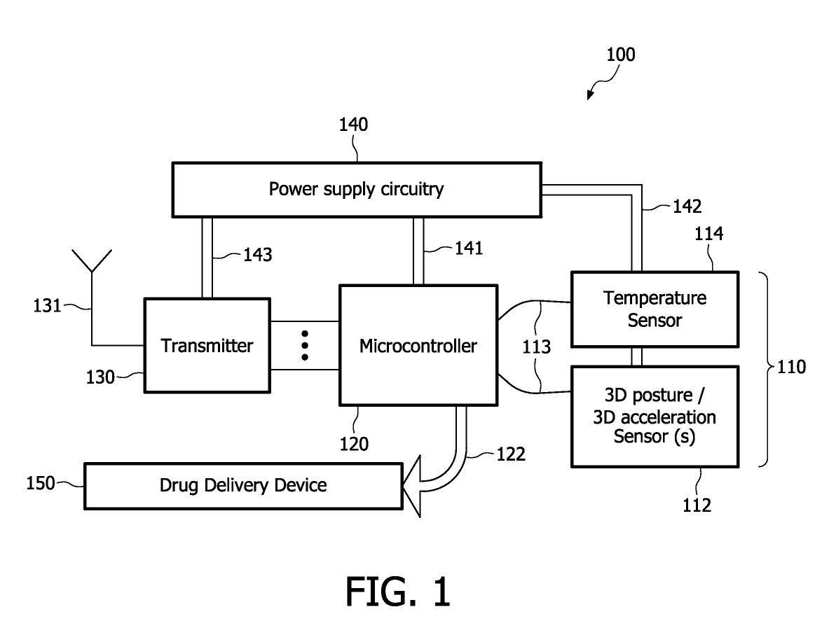

FIG. 1 shows a simplified block diagram 100 of a drug delivery system. The drug delivery system 100 comprises a measurement device 110, which includes a temperature sensor 114 and a sensor 112 for detecting the posture and/or the acceleration of at least a part of a patient's body. By means of a data connection 113 the sensors 112 and 114 are coupled with a control unit 120. The control unit is realized by means of a microcontroller 120.

The control unit 120 is connected to a drug delivery device 150 also by means of a wired or a wireless data connection 122, which allows for controlling the drug delivery device 150 based on measurement signals being provided by the measurement device 110. These signals are indicative for the current activity status of the patient.

The drug delivery system 100 further comprises a transmitter unit 130, which is coupled to the control unit 120. The transmitter unit 130 and the control unit 120 may be formed as separate devices or integrally as a single device. The transmitter unit 130 is connected to an antenna 131, which allows for a wireless communication between the control unit 120 and a not depicted external system. The external system may be a medical alert system or any other medical system, which allows for storing and/or evaluating data representing the operation of the drug delivery system 100. In particular, the received measurement data may be evaluated for instance by a physician in order to obtain detailed information about the status of the patient's disease.

The drug delivery system 100 further comprises a power supply circuitry 140. The power supply circuitry 140 comprises a power supply source such as a battery. The power supply circuitry 140 is connected (a) to the measurement device 110 by means of a power supply connection 141, (b) to the microcontroller 120 by means of a power supply connection 142 and (c) to the transmitter 130 by means of a power supply connection 143.

According to the embodiment described here the power supply circuitry 140 further comprises a power supply source monitoring circuit. This circuit is adapted to monitor the state of the power supply source being used for powering the measurement device, the control unit and/or the drug delivery device.

It has to be mentioned that all or at least some components of the drug delivery system 100 shown in FIG. 1 may be implanted within the patient's body. This holds in particular for the drug delivery device 150.

In an already working and tested prototype of the drug delivery system 100 all components except the drug delivery device 150 are realized in a miniaturized form. The miniaturized form has a cylindrical shape and a volume of about 0.26 ccm. Thereby, a hot temperature conductor thermistor is used as a temperature sensor. For the acceleration sensor 112 the 3-axis Accelerometer ADXL330 (iMEMS®) having a measurement range between −3 g and +3 g and an analog output has been used. The control unit 120 has been realized by a 8 Bit RISC controller with 4 kByte ROM. Further, a 12 bits ADC-converter and an on-board voltage reference has been used.

Apart from the temperature and acceleration measurement the described prototype is capable also of monitoring the battery voltage. The overall data stream contains 16 bytes and includes the battery voltage reading (2 bytes), the data representing the temperature (2 bytes), the data representing the 3D acceleration (6 bytes), one synchronization byte, one parity check byte and a unique identification code (4 bytes).

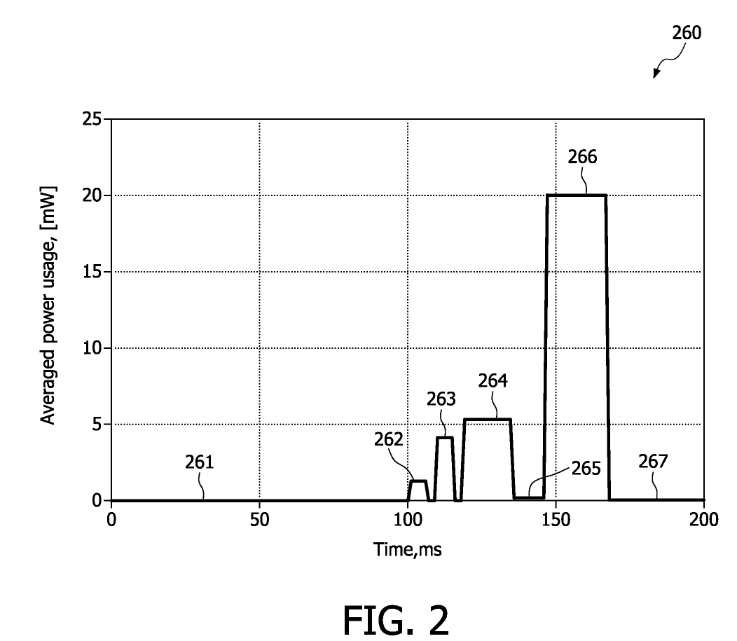

FIG. 2 shows a diagram 260 depicting the time dependency of the power consumption of a preferable operation mode of the drug delivery system 100. The power consumption of the drug delivery device 150 is initially not taken into account.

In order to reduce the power consumption the drug delivery system 100 is in a power down mode 261 most of the time. The whole process of measuring, calculating and sending the data takes about 70 milliseconds. In order to reduce the effective power consumption the system is in an idle mode 265 in between the reading cycles and the data transmission 266. This holds for (a) the time interval in between a voltage measurement 262 and a temperature measurement 263, (b) the time interval in between the temperature measurement 263 and a 3D acceleration measurement 264 and (c) the time interval in between the 3D acceleration measurement 264 and the data transmission 266. For sake of clarity only the idle mode 265 in between the 3D acceleration measurement 264 and the data transmission 266 is denominated by the reference numeral 265. For the remaining time, the system operates in power-down mode 267. This leads to an extreme low total power consumption.

In the following a realistic estimation regarding the expected lifetime of a battery powering the above-described prototype will be given: If the measurement data is obtained and sent every 10 seconds, the averaged power consumption of the measuring device of the system amounts to about 75 μW This means, if the system is powered with a standard Lithium battery (type CR1225) having a power capacity of approximately 150 mWh (voltage=3 V, amperage capacity=48 mAh), the measurement unit 110, the control unit 120 and the transmitter unit 130 of the drug delivery system 100 are capable of measuring and sending data for about 85 days.

For driving a motor of the drug delivery device the necessary current consumed per step measures 5 mA. Each step takes a period of about 2 seconds and the stepper motor takes for one turn 20 steps. The pitch of the screw rod is 0.2 mm. If the drug reservoir is 15 mm long then the energy required to empty the reservoir equals

(15/0.2)*20*5*10⁻³*3*2=45 J.

If the entire drug delivery system 100 comprising the measurement device 110, the control unit 120, the transmitter unit 130 and the actuator parts of the drug delivery device 150 are powered by the same standard Lithium battery (type CR1225) having an amperage capacity of 48 mAh corresponding to 48 *10⁻³ mAh*3V*3600 s/h=518 J, the energy being left for sensing and data transmitting is about 473 J. This corresponds to 44 mAh=473 J/(3V*3600 s/h)) or 130 mWh. This leads to a total lifetime of the whole drug delivery system 100 of 72 days.

It has to be mentioned that the measurement device 110 and the drug delivery device 150 may be integrated in one device or may perform as two separate devices. In the latter case the drug delivery device 150 may be implantable and the measurement device 110 may designed for being attached externally to the body of a patient.

As has already been mentioned above, the drug delivery device 150 may be realized by one of the following devices or by a drug delivery device, which is from a technical point of view related to one of the following devices: a device containing liquid medication propelled by for example a step-motor based drug pump, a miniature piezo-driven drug pump, a drug delivery system with switchable membranes, an implantable drug delivery system with external magnetic, electrochemical or electro-osmotic actuation, a spring driven pump system or a device containing a solid type of medication, that may be delivered as dry powder or dispersed into gas or liquid and then administered to the body. The pump may be an external (ambulatory) or internal (implantable) drug delivery system.

To prevent processes such as encapsulation and/or to prevent body fluids entering into the drug reservoir during the period the pump mechanism/actuator is turned off, the implementation of a constant releasing drug may be suitable. Preferably, such a constant drug release is not battery powered.

For instance when treating a hemophilia A patient both the power consumption of the drug delivery system and the required amount of drug can be reduced significantly if the drug delivery system is switched of during a time period, wherein the body of the patient is in a very inactive state. Such a period is in particular the night, when the patient is asleep and when the risk of getting hurt is extremely low.

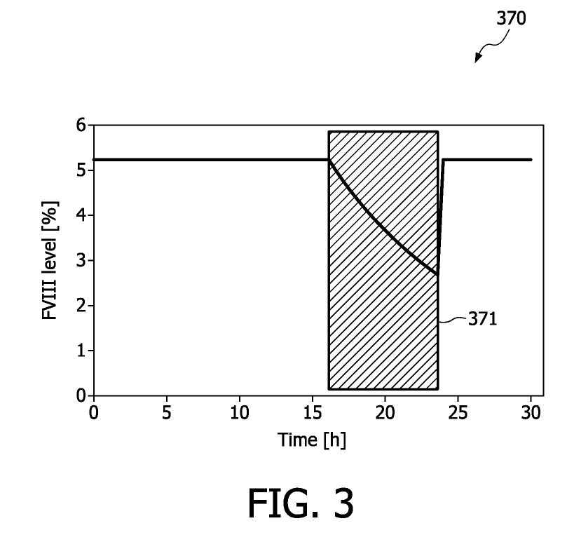

FIG. 3 shows a diagram 370 depicting the time dependency of a patient's blood drug level during an intermediate halt of a drug release. According to the exemplary embodiment described here, the patient is treated with FVIII. As can be seen from FIG. 3, during the nocturnal period, wherein the drug release is halted, the FVIII level within the patient's blood is decaying. As soon as the time window with no drug release is finished, the FVIII level increases again to the value being desired for normal activity of the patient.

Since the main power consummators are the drug delivery pump, the measurement device providing the sensors signals and eventually a wireless data transmission, one can achieve significant savings in power consumption if switching off any of the parts when their operation is not required. In addition to a reduction in power consumption, there is a reduction in amount of medication. The percent wise reduction is independent on prophylaxis trough level (5% in diagram 370) and also independent on the volume of distribution of the patient. However, the percent wise reduction strongly depends on the half-life of the drug within the patient's blood. For hemophilia A, typical drug amount reductions are in the range 7-10%. When looking at the reduction in FVIII medication on an annual basis, a patient having a prophylaxis level of 2% will still save in the order of 7,500 International Units when the drug release is turned off during the sleep. A patient on a prophylaxis level of 5% will save in the order of 20,000 International Units within one year. Therefore, the described drug delivery system, which takes into account the activity status of the patient, is capable of saving a lot of money when treating for instance patients suffering from hemophilia A disease.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state: It is described a drug delivery system 100 that is adapted to infuse at least one drug in a profile related to the status of a patient. The system comprises sensors 110, dedicated electronics 140 and software to monitor the patient's activity and maintain control over the drug-release profile. A control unit 120 of the system 100 is adapted to temporarily set at least one component 110, 120, 150 of the system 100 in an idle mode 265. This will result in particular in a reduction of power required by the system 100. The described system 100 enables a personalized treatment of patients by monitoring their individual activity status and coupling this information to a drug delivery following appropriate delivery profiles. The system 100 may be provided with a memory in order to store a time dependence of activity data. This time dependence may be used by physicians to monitor, support and adapt their therapy for the patients. The system 100 may be realized as a closed-loop system for monitoring and treatment of various diseases such as hemophilia A and Parkinson.

### LIST OF REFERENCE SIGNS

100 block diagram of drug delivery system
110 measurement device
112 3D posture sensor/3D acceleration sensor
113 data connection (wired or wireless)
114 temperature sensor
120 control unit
122 control line (wired or wireless)
130 transmitter unit
131 antenna
140 power supply source/battery/battery monitoring circuit
141 power supply connection
142 power supply connection
143 power supply connection
150 drug delivery device
260 time diagram depicting power consumption
261 power down
262 voltage measurement
263 temperature measurement
264 3D acceleration measurement
265 drug delivery system idle
266 data transmission
267 power down
370 time diagram depicting drug (FVIII) level
371 time window with no drug release

1. A system for delivering at least one drug to the body of a patient, the system (100) comprising
a measurement device (110) for measuring at least one measurement value, which is indicative for the status of the patient,
a control unit (120), which is coupled to the measurement device (110) and which is adapted to determine a control signal based on the measurement value, and
a drug delivery device (150), which is coupled to the control unit (120) and which is adapted to deliver the drug to the patient's body based on the control signal, wherein
the control unit (120) is adapted to temporarily set at least one component (110, 120, 150) of the system (100) in an idle mode (265).

2. The system according to claim 1, wherein
the control unit (120) is adapted to control a sampling rate of the measurement device (110).

3. The system according to claim 1, wherein
the measurement device (110) comprises an acceleration sensor (112), in particular a three-dimensional acceleration sensor.

4. The system according to claim 1, wherein
the measurement device (110) comprises a magnetic field sensor.

5. The system according to claim 1, wherein
the measurement device (110) comprises a temperature sensor (114).

6. The system according to claim 1, wherein
the measurement device (110) comprises a skin impedance sensor.

7. The system according to claim 1, further comprising
a memory for storing a plurality of measurement values, which are indicative for the status of the patient, wherein the measurement values have been acquired at different times.

8. The system according to claim 1, wherein
the measurement device (110), the control unit (120) and/or the drug delivery device (150) is implantable into a patient's body.

9. The system according to claim 1, wherein
the control unit (120) is adapted to allow for an interventional control ability.

10. The system according to claim 1, further comprising
a radio transmitter (130), which is coupled to the control unit (120).

11. The system according to claim 1, further comprising
a battery monitoring circuit (140), which is adapted to monitor the state of a battery being used for powering the measurement device (110), the control unit (120) and/or the drug delivery device (150).

12. The system according to claim 1, wherein
the drug delivery device (150) is adapted to deliver liquid type of medication.

13. The system according to claim 1, wherein
the drug delivery device (150) is adapted to deliver solid type of medication.

14. A method for delivering at least one drug to the body of a patient by using a system as set forth in claim 1, the method comprising
measuring at least one measurement value, which is indicative for the status of the patient,
determining a control signal based on the measurement value,
delivering the drug to the patient's body based on the control signal, and
temporarily setting at least one component (110, 120, 150) of the system (100) in an idle mode (265).
